(19) United States
(12) Patent Application Publication
(10) Pub. No.: US 2011/0287080 A1
(43) Pub. Date: Nov. 24, 2011

(54) COATING II

(75) Inventors: David Franklin Farrar, York (GB); Kadem Gayard Al-Lamee, Leeds (GB); Jeffrey Nmi Motley, Sheffield (GB)

(73) Assignee: SMITH & NEPHEW PLC, Memphis, TN (US)

(21) Appl. No.: 13/125,010

(22) PCT Filed: Oct. 21, 2009
(86) PCT No.: PCT/GB2009/002501

§ 371 Date: Aug. 12, 2011

(30) Foreign Application Priority Data
Oct. 21, 2008 (GB) 0819296.5

(51) Int. Cl. A61K 9/00; A61P 9/00; B05D 3/00; A61K 31/436; A61K 38/14

(52) U.S. Cl. 424/426; 514/291; 514/3.1; 424/422; 427/2.1

(57) ABSTRACT

The invention relates to a coating composition for an implantable medical device, a method of coating a medical device and a device coated with the composition.

### RELATED APPLICATIONS

This application claims priority from UK provisional application No. 0819296.5 entitled “Coating II” filed on Oct. 21, 2008, which is herein incorporated in its entirety.

### FIELD OF THE INVENTION

The invention relates to a coating composition for an implantable medical device, a method of coating a medical device and a device coated with the composition.

### BACKGROUND TO THE INVENTION

Stents are small expandable metal tubes that are implanted in arteries to keep them open in patients whose vessels have become narrowed due to coronary artery disease, the most common cause of death in the Western World. Bare metal stents sometimes become re-narrowed again (restenosis) requiring a re-intervention procedure to re-open them.

A drug-eluting stent, sometimes referred to as a “coated” or “medicated” stent, is a normal bare metal stent that has been coated with a polymer containing a pharmacologic (drug) that is known to interfere with the process of restenosis (re-narrowing). Restenosis has a number of causes; it is a very complex process and the solution to its prevention is equally complex. However, in the data gathered so far, the drug-eluting stent has been extremely successful in reducing restenosis from the 20-30% range to single digits.

After stent implantation, in addition to aspirin, the patient must take an anti-clotting or anti-platelet drug, such as clopidogrel or ticlopidine (brand names Plavix and Ticlid) for six or more months after stenting, to prevent the blood from reacting to the new device by thickening and clogging up the newly expanded artery (thrombosis). Ideally a smooth, thin layer of endothelial cells (the inner lining of the blood vessel) grows over the stent during this period and the device is incorporated into the artery, reducing the tendency for clotting.

Despite the clinical and commercial success of drug-eluting stents, recent evidence has suggested that they result in higher rates of late stage thrombosis, a clot formation at the end of the stent, which, in over 50% of cases, results in either an acute heart attack or death. Many physicians believe that the clot formations are due to the delayed healing of the vessel caused by the cytotoxic drugs that coat the stent or by some biostable polymers that are used to deliver the drug. In response to these concerns some physicians are defaulting to using bare metal stents, where late stent thrombosis is not seen as much of a problem.

Recently, industry has been working on replacing the biostable polymer coatings used in first generation drug-eluting stents with bioresorbable polymers. Most of the current bioresorbable coatings used for the drug-eluting stents are based on poly(lactide), poly(glycolide) or co-polymers of the two (poly(lactide-co-glycolide)). It is difficult to control the drug-elution profile with these polymers, one problem being a “burst release” of the drug that gives a high initial release of drug followed by a slow release, whereas what is desired is a more steady and sustained release.

Another problem with coatings on stents is the adhesion of the polymer to the metal surface. Most polymer coatings will not adhere strongly to a normal metal surface and the coating may delaminate when the stent is deployed inside the vessel.

As a result, there is a requirement for a new generation of drug-eluting stents which have bioresorbable coatings in place of the current stable (non-resorbable) coatings.

In this way the stent can deliver its payload of a drug from the coating but once the drug has been delivered the coating resorbs so that the stent effectively reverts to a bare metal stent, with lower chance of late stent thrombosis.

### SUMMARY OF THE INVENTION

The coating composition of the invention is a drug-eluting bioresorbable coating. The composition of the coating allows control of the elution profile of the drug as well as allowing strong adhesion of the coating to a metal surface and resistance to damage.

According to a first aspect of the invention there is provided a bioresorbable coating composition for an implantable medical device, wherein the composition comprises a co-polymer of a lactide, a glycolide and s-caprolactone and at least one drug and/or bioactive agent.

According to a second aspect of the invention there is provided an implantable medical device coated with the bioresorbable coating composition according to the first aspect of the invention.

According to a third aspect of the invention there is provided a method of applying a bioresorbable coating composition to an implantable medical device comprising the steps of;
i. providing an implantable medical device,
ii. applying a coating of the bioresorbable coating composition according to the first aspect of the invention to at least part of a surface of the device.

According to a fourth aspect of the invention there is provided a method of using a device which is coated with the bioresorbable coating composition according to the first aspect of the invention, wherein the method comprises the step of implanting the device in a non-human animal body or human body.

According to a fifth aspect of the invention there is provided a vehicle, for carrying a drug, wherein the vehicle is defined as the composition of the present invention.

According to a still further aspect of the invention, there is provided a bioresorbable coating composition, implantable medical implant, vehicle for carrying a drug and methods of producing thereof and methods of use thereof as substantially herein described with reference to the accompanying Examples and Figures.

In embodiments of the invention the composition comprises a glycolide at between 30-45% by weight of the polymer component of the composition.

In embodiments of the invention the composition comprises a glycolide at between 5-15% by weight of the polymer component of the composition.

In embodiments of the invention the composition comprises a glycolide at about 10% by weight of the polymer component of the composition.

In embodiments of the invention the composition comprise s-caprolactone at between about 10-20% by weight of the polymer component of the composition.

In embodiments of the invention the composition comprises ε-caprolactone at about 12.5% by weight of the polymer component of the composition.

In embodiments of the invention the composition comprises a glycolide at between about 10% by weight of the polymer component of the composition and ε-caprolactone at about 12.5% by weight of the polymer component of the composition

In embodiments of the invention the composition the lactide is D,L-lactide.

In embodiments of the invention the composition comprises a blend of co-polymers. For example the second co-polymer of the blend is also a co-polymer of a lactide, a glycolide and ε-caprolactone.

In a specific embodiment of the invention the bioresorbable coating composition comprises a blend of a first co-polymer comprising 47.5% D,L-lactide, 40% glycolide and 12.5% ε-caprolactone and a second co-polymer comprising 57.5% D,L-lactide, 30% glycolide and 12.5% ε-caprolactone.

A “bioactive agent” or “drug” is herein defined as any biological and/or chemical substance used in the treatment, cure, prevention, or diagnosis of disease or used to otherwise enhance physical or mental well-being. Examples of a suitable drug type for incorporation into the coating composition of the present invention include, an anti-inflammatory agent, a cytotoxic agent, an angiogenic agent, an osteogenic agent, an immunosuppressant, an anti-clotting agent, an anti-platelet agent, an antimicrobial or an antibiotic.

Suitable anti-platelet agents include clopidogrel, sold as Plavix™ by Bristol Myers-Squibb and ticlopidine, sold as Ticlid™ by Sanofi-Aventis. Suitable anti-clotting agents include heparin.

Suitable immunosuppressants include rapamycin, also known as Sirolimus available from AG Scientific Inc.

Suitable antibiotics include gentamicin and vancamycin.

In embodiments of the invention the composition comprises rapamycin at about 5%, 10%, 15%, 20%, 25%, 30%, 35%, 40%, 45% or 50% by weight of the composition.

In a specific embodiment of the invention the composition comprises rapamycin at between about 20-30% by weight of the composition.

In a further specific embodiment of the invention the composition comprises rapamycin at about 25% by weight of the composition.

A bioactive agent is herein defined as any agent that has an effect on, interaction with, or response from living tissue. For example, an antibody or a cell (e.g a stem cell).

The composition can further comprise at least one additive which is an acid or a derivative thereof selected from the group consisting of hexanoic acid, octanoic acid, decanoic acid, lauric acid, myristic acid, crotonic acid, 4-pentenoic acid, 2-hexenoic acid, undecylenic acid, petroselenic acid, oleic acid, erucic acid, 2,4-hexadienoic acid, linoleic acid, linolenic acid, benzoic acid, hydrocinnamic acid, 4-isopropylbenzoic acid, ibuprofen, ricinoleic acid, adipic acid, suberic acid, phthalic acid, 2-bromolauric acid, 2,4-hydroxydodecanoic acid, butyric acid, monobutyrin, 2-hexyldecanoic acid, 2-butyloctanoic acid, 2-ethylhexanoic acid, 2-methylvaleric acid, 3-methylvaleric acid, 4-methylvaleric acid, 2-ethylbutyric acid, trans-beta-hydromuconic acid, isovaleric anhydride, hexanoic anhydride, decanoic anhydride, lauric anhydride, myristic anhydride, 4-pentenoic anhydride, oleic anhydride, linoleic anhydride, benzoic anhydride, poly(azelaic anhydride), 2-octen-1-yl succinic anhydride and phthalic anhydride.

Aptly the composition contains the additive in an amount which is not more than 10%, typically not more than 5%, and even more typically not more than 2% by weight of the composition.

The amount of the additive chosen will also depend upon the rate of degradation desired. In vivo degradation occurs firstly by hydrolytic scission of the polymer chains resulting in the formation of units of increasingly smaller molecular weight until only substantially monomers remain. Thereafter, the monomers are metabolized and absorbed into the body. It is only in the last stages of degradation that mass loss occurs.

It has been found that a particularly suitable additive for use in the invention is lauric acid. This may be employed as the acid per se or, if desired, as a derivative, for example as the anhydride.

Advantageously compositions will contain lauric acid a derivative thereof in an amount not more than 10%, more typically not more than 5%, and even more typically not more than 2% by weight of the composition.

The bioresorbable coating composition of the invention may be applied directly to the implantable medical device. However, most polymeric coatings will not adhere strongly to a normal metal surface and the coating has a tendency to de-laminate. In embodiments of the invention the bioresorbable coating of the invention is chemically and/or physically coupled to the surface of the device.

In embodiments of the invention “functionalisation” of the metal surface improves the adhesion of the polymeric coating to the surface of the medical device and minimises the risk of delamination. This functionalisation provides a “chemical bridge” between the metal surface and the polymeric coating.

In the first stage of the functionalisation process a chemical is chosen that reacts well with the inherent functionality of the metal surface. The chemical preferably reacts with the oxides, hydroxides, epoxide or any other surface oxide on metallic surfaces to form strong bonds whilst leaving the rest of the molecule free to react with other species.

Suitable chemicals for use in the first reaction step include alkoxysilanes of the formula (RO)₃Si(R¹X) wherein R represents methyl or ethyl and R¹ represents C₂-C₁₀ alkyl in which one or more methylene groups may be replaced by —NH— or —O—, C₂-C₁₀ cycloalkyl or cycloalkylalkyl, C₂-C₁₀ aralkyl or monocylic or bicyclic aryl and X represents amino, hydroxyl, carboxylic acid or acid anhydride. Preferably R¹ represents C₂-C₁₀ alkyl in which one or more methylene groups is optionally replaced by —NH— and X represents —NH₂, and an example of a suitable priming agent is N-[3-(trimethoxysilyl)propyl]ethylenediamine.

In the second stage, another chemical is chosen which reacts readily with the functional/reactive groups of the first chemical and which also has a functional group which can react with oxygen containing groups in the polymer, for example, hydroxyl, methoxy and ethoxy groups. A strong bond is therefore formed between the two molecules and the polymer is coupled to the functionalised surface. In this way a strong chemical bond is achieved between the functionalised surface and the polymer, improving the adhesion of the polymer to the metal surface. Any chemical with an alkoxysilyl group on one end and an isocyanate on the other end is suitable for use in the second reaction step. An example of an appropriate chemical is 3-(triethoxysilyl)propylisocyanate.

FIGS. 1 and 2 illustrate the functionalisation process.

In embodiments of the invention the bioresorbable coating composition of the first aspect of the invention contains —OH groups to react with the triethoxy groups from the second functionalisation step.

In further embodiments of the invention an intermediate coating composition is provided between at least part of the surface of the device and the bioresorbable coating composition of the first aspect of the invention to physically “tie” the coating composition in place. This intermediate coating composition can be referred to as a “primer coat” or a “tie coat”. This composition comprises a lower molecular weight polymer of similar or identical chemical composition to the polymeric component of the bioresorbable coating composition of the first aspect of the invention. The polymeric composition of the “tie coat” can be any polymer which is capable of forming a strong adhesion with the polymeric component of the bioresorbable coating composition of the first aspect of the invention.

In embodiments of the invention the primer coat comprises a co-polymer of a lactide and a glycolide, for example poly(D,L-lactide-co-glycolide), specifically in which the ratio of D,L-lactide to glycolide is 50:50 and the molecular weight of the copolymer is between about 5-15 k.

The primer coat may be applied directly to at least part of the surface of the device and then the bioresorbable coating composition according to the first aspect of the invention is applied so as to overlay at least part of the primer coat.

In alternative embodiments of the invention the surface of the device may be chemically functionalised as described above, with the primer coat being applied to this'functionalised surface and then the bioresorbable coating composition of the first aspect of the invention being applied so as to overlay at least part of the primer coat The elution profile of the drug can be further controlled by the use of undrugged top-coat covering, wholly or partially the bioactive/drug-containing layer (i.e the bioresorbable coating composition according to the first aspect of the invention). This composition can comprise a polymer of similar or identical chemical composition to the bioresorbable coating composition according to the first aspect of the invention. For example, the top coat may be a copolymer of lactide, glycolide and ε-caprolactone or it may be a copolymer of D,L-lactide and glycolide.

The top coat can form between about 1%-50%, or about 1%-45%, or about 1%-40%, or about 1%-35%, or about 1%-30%, or about 1%-25%, or about 1%-20%, or about 1%-15%, or about 1%-10%, or about 1%-5%, or about 5%-10%, or about 5%-15%, or about 5%-20%, or about 10%-20%, or about 15%-20% of the total coating weight (e.g: (i) bioresorbable coating composition according to the first aspect of the invention+top coat or (ii) primer coat+bioresorbable coating composition according to the first aspect of the invention+top coat)

In further embodiments of the invention the polymer component of all of the polymeric coatings may comprise a blend of two or more polymers that themselves comprise copolymers of lactide, glycolide and s-caprolactone, for example in the ratios outlined above.

Medical devices for which this coating technology may be advantageous, include but are not limited to stents, orthopaedic implants, dental implants and maxillo-facial implants.

Examples of stents to which the drug-eluting bioresorbable coating of the invention can be applied include coronary stents, carotid stents, aortic stents, renal stents and venous stents. Other examples of stents include peripheral stents.

Examples of orthopaedic implants to which the drug-eluting bioresorbable coating can be applied include reconstructive and trauma products, for example, components of hip replacement, components of knee replacements, fracture plates, screws, pins, external fixation plates, intramedullary nails, interference screws, suture anchors.

Examples of maxillo-facial implants to which the drug-eluting bioresorbable coating of the invention can be applied include plates, screws and meshes.

Further areas of applicability of the present invention will become apparent from the detailed description provided hereafter. It should be understood that the detailed description and specific examples, while indicating the preferred embodiment of the invention, are intended for purpose of illustration only and are not intended to limit the scope of the invention.

### BRIEF DESCRIPTION OF THE DRAWINGS

The accompanying drawings, which are incorporated in and, form part of the specification, illustrate the embodiments of the present invention and together with the written description serve to explain the principles, characteristics, and features of the invention. In the following drawings:

FIG. 1: Illustrative chemistry

FIG. 2: Schematic of surface functionalisation

FIG. 3: Percentage of rapamycin released from PLGC polymers (PLGC1, 4, 7, 10, 18) into PBS at 37° C.

FIG. 4: Percentage of rapamycin released from PLGC polymers (PLGC12, 13, 14, 15) into PBS at 37° C.

FIG. 5: Percentage of rapamycin released from PLGC polymers with undrugged PLGA top-coats (10% of total coat weight) into PBS at 37° C.

FIG. 6: Percentage of rapamycin released from 60/40 (PLGC4/8) polymers with 60/40 (PLGC4/8) undrugged top coats of varying thicknesses.

FIG. 7: Percentage of rapamycin released from blended PLGC polymers with top coats into PBS at 37° C.

FIG. 8: Percentage of rapamycin released from blended PLGC polymers (PLGC4/PLGC8) with PLGC4 top coats into PBS at 37° C.

FIG. 9: Percentage of rapamycin released from blended PLGC polymers containing lauric acid into PBS at 37° C.

FIG. 10: Adhesion strength of bioresorbable polymer on functionalised and unfunctionalised metal surfaces.

FIG. 11: The polymer-coated stent was placed onto the balloon section of a suitably sized commercially available PTCA catheter and crimped by hand. The catheter was fed down a tortuous path in PBS solution to mimic the passage of the stent and catheter in the human arteries.

FIG. 12: In vitro rapamycin release from PLGC coated stents in PBS at 37° C. in which the glycolide forms about 10% of the polymeric composition of the coating.

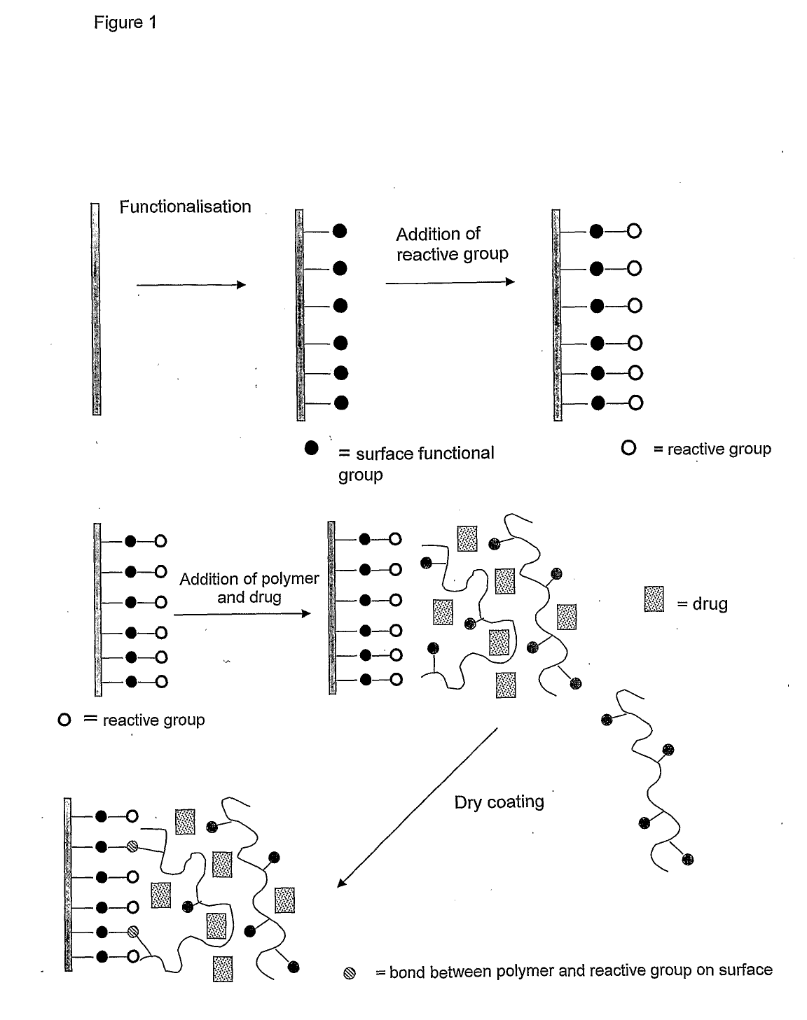

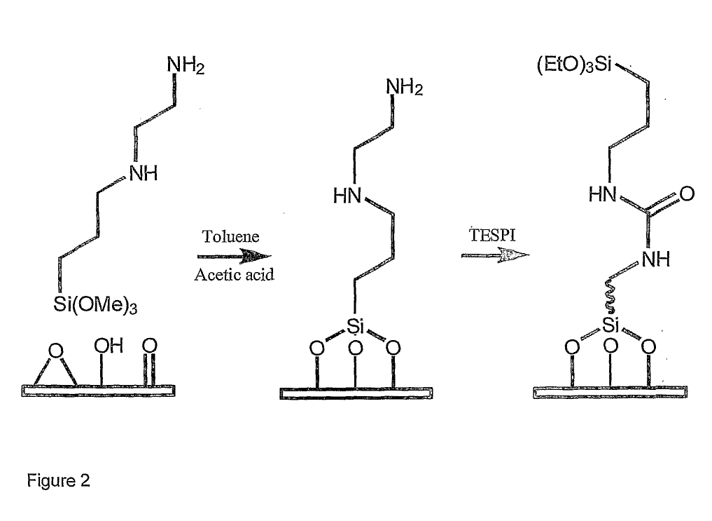

### DETAILED DESCRIPTION OF THE INVENTION

### Example 1

### Polymerisation

### i) Materials

### DL-lactide (Purac)

### Glycolide (Purac)

### ε-Caprolactone (Aldrich)

Tin(II) chloride dihydrate (Aldrich)
Diethylene glycol (Aldrich)

### Dichloromethane (Fisher)

### ii) Initiator-Catalyst System

Tin(II) chloride dihydrate (0.5 g) was added to diethylene glycol (1.46 g) and heated gently to dissolve the tin chloride

iii) Polymer Production

D,L-lactide, glycolide and s-caprolactone were weighed into a Wheaton vial, total weight of 40 g. 10 microlitres of tin chloride in diethylene glycol was added to each vial together with 100 microlitres of diethylene glycol. The vials were placed in an oil bath set at 150° C. and stirred until the viscosity became so great that the magnetic follower wouldn't go round after which time the vials were placed in the oven at 150° C. and left for ˜16 hours. To remove the polymer from the vials dichloromethane was added and the vials rolled until the polymer dissolved. The polymer solution was poured onto siliconised paper and the dichloromethane evaporated off. To remove residual monomer the polymers were melted under vacuum to boil the monomer off.

### Characterisation of Polymers

### (i) Molecular Weight

Molecular weight distributions were determined by conventional Gel Permeation Chromatography (GPC) in chloroform. Column calibration was achieved using narrowly disperse polystyrene standards

Samples were dissolved in GPC grade chloroform with 0.1% v/v toluene as flow rate marker.

### Column: PLgel MIXED-D 300×7.5 mm

Mobile phase: Chloroform (GPC grade)
Flow rate: 1 mL/min
Column temp: 30° C.
Sample conc: approx 2 mg/mL
Injection volume: 100 μL

The results are shown in Table 1.

ii) Glass transition temperature

The glass transition temperatures of the polymers were measured by Differential Scanning calorimetry using a Perkin Elmer DSC7. The heating rate was 10° C./min. The results are shown in Table 1.

The ratios of the lactide, glycolide and ε-caprolactone in the different are shown in Table 1.

TABLE 1
Properties of polymers

| Ref | Composition (%) | | | | Molecular weight | | |
|---|---|---|---|---|---|---|---|
| Code | Lactide | Glyolide | Caprolactone | Tg | Mw | Mn | PD |
| PLGC1 | 40 | 40 | 20 | 17.7 | 41600 | 24900 | 1.67 |
| PLGC2 | 45 | 45 | 10 | n/a | n/a | n/a | |
| PLGC3 | 42.5 | 42.5 | 15 | 29.8 | 52600 | 24600 | 2.13 |
| PLGC4 | 47.5 | 40 | 12.5 | 28.9 | 36100 | 20700 | 1.74 |
| PLGC5 | 45 | 40 | 15 | 30.2 | 59800 | 32100 | 1.86 |
| PLGC6 | 42.5 | 40 | 17.5 | 30.6 | 64400 | 32300 | 1.99 |
| PLGC7 | 47.5 | 37.5 | 15 | 24.9 | 46900 | 25300 | 1.85 |
| PLGC8 | 57.5 | 30 | 12.5 | 38 | 56500 | 29900 | 1.89 |
| PLGC9 | 47.5 | 40 | 12.5 | 27.8 | 60508 | 26887 | 2.26 |
| PLGC10 | 57.5 | 30 | 12.5 | 32 | 70024 | 31939 | 2.19 |
| PLGC11 | 50 | 37.5 | 12.5 | 31.4 | 38700 | 20500 | 1.89 |
| PLGC12 | 52.5 | 35 | 12.5 | 25.9 | 38100 | 21200 | 1.8 |
| PLGC13 | 55 | 32.5 | 12.5 | 29.1 | 50300 | 25600 | 1.96 |
| PLGC14 | 47.5 | 40 | 12.5 | 23 | 33600 | 18400 | 1.83 |
| PLGC15 | 57.5 | 30 | 12.5 | 33.9 | 41500 | 21600 | 1.92 |
| PLGC16 | 40 | 40 | 20 | n/a | 38000 | 19300 | 1.97 |
| PLGC17 | 47.5 | 37.5 | 15 | n/a | 44200 | 19100 | 2.31 |
| PLGC18 | 47.5 | 35 | 17.5 | n/a | 36400 | 15000 | 2.43 |

### Example 2

### i) Materials

Sodium hydrogen carbonate: Sigma Aldrich
N[3-(trimethoxysilyl)propyl]ethylenediamine (TMSPEA): Sigma Aldrich
Glacial acetic acid: Sigma Aldrich
3-(triethoxysilyl)propyl isocyanate (TESPI): Sigma Aldrich

### Poly(D,L-lactide-co-glycolide) (PLGA1) 50:50, Mw 5-15 k: Sigma Aldrich

### Poly(D,L-lactide-co-glycolide) (PLGA2) 50:50, Mw 10 k: Durect

Lauric acid (LA): Sigma Aldrich
Sodium dodecyl sulphate (SDS): Sigma Aldrich
Rapamycin: LC laboratories

### Phosphate Buffered Saline (PBS): Sigma Aldrich

### Solvents: Sigma Aldrich

ii) Coating of Stents with Polymer Composition

### Stage 1: Cleaning

Commercially available stainless steel stents were cleaned by sonication for 15 minutes in a 7.5% w/w solution of aqueous sodium hydrogen carbonate, rinsing in deionised water, sonication for 15 minutes in 2-propanol and sonication for 15 minutes in deionised water. The samples were then dried at 100° C. for 16 hours, followed by drying at 50° C. for 30 minutes.

### Stage 2: Functionalisation

2.5 ml glacial acetic acid solution in toluene (1.0% w/w, 0.4 mmol) was added to 200 ml toluene followed by 1.6 ml TMSPEA and mixed. The samples were removed from the 50° C. oven and immersed in the solution for 5 minutes. The samples were removed and kept at 50° C. for 20 hours.

The samples were rinsed in a series of solvents by rotating sequentially for 15 minutes in each of toluene, methanol, deionised water, methanol and deionised water. Finally, the samples were rinsed for 5 minutes in methanol and then dried at 50° C. for 2 hours.

Anhydrous toluene was added, under nitrogen, into a measuring cylinder being purged with nitrogen. Enough TESPI was added to give a 4% v/v solution in toluene. The samples (dried at 50° C. for 15 minutes and allowed to cool for two minutes before use) were immersed in the solution on a holder and rotated under nitrogen for 15 minutes. The samples were then rinsed in anhydrous toluene under nitrogen and dried under vacuum for 16 hours.

### Stage 3: Applying a Primer Coat

The stents were attached to a mandrel and coated with a primer solution containing 0.5% w/w PLGA1 in CHCl₃ on a Sonotek MediCoat Benchtop Coater. The parameters used were: 0.075 ml/min flow rate, 0.8 W ultrasonic power, 2 passes, 40 rpm rotation, 0.13 cm/s horizontal travel and 25 mm from stent to spray head. After priming, the stents were left for 16 hours at 100° C.

### Stage 4: Preparing the Drug-Containing Biresorbable Coating

Solutions were prepared by dissolving the polymers listed in Table 1 in CHCl₃ with 25% (by weight of the solid polymer) rapamycin.

### Stage 5: Coating

After cooling for 5 minutes, the stents were attached to a mandrel and coated on the Sonotek MediCoat Benchtop Coater with a 0.5% w/w solution in CHCl₃ of polymer/drug (75/25). The parameters used were: 0.09 ml/min flow rate, 1.0 W ultrasonic power, 16 passes, 40 rpm rotation, 0.13 cm/s horizontal travel and 25 mm from stent to spray head. After coating, the stents were dried under vacuum for 16 hours at 40° C.

### Stage 6: Elution Testing

The stents were released into phosphate buffered saline solution (PBS) at 37° C. and the elution monitored by UV/vis spectroscopy. Fresh buffer solution was added after each reading and the cumulative absorbance at 279 nm was recorded.

### Results

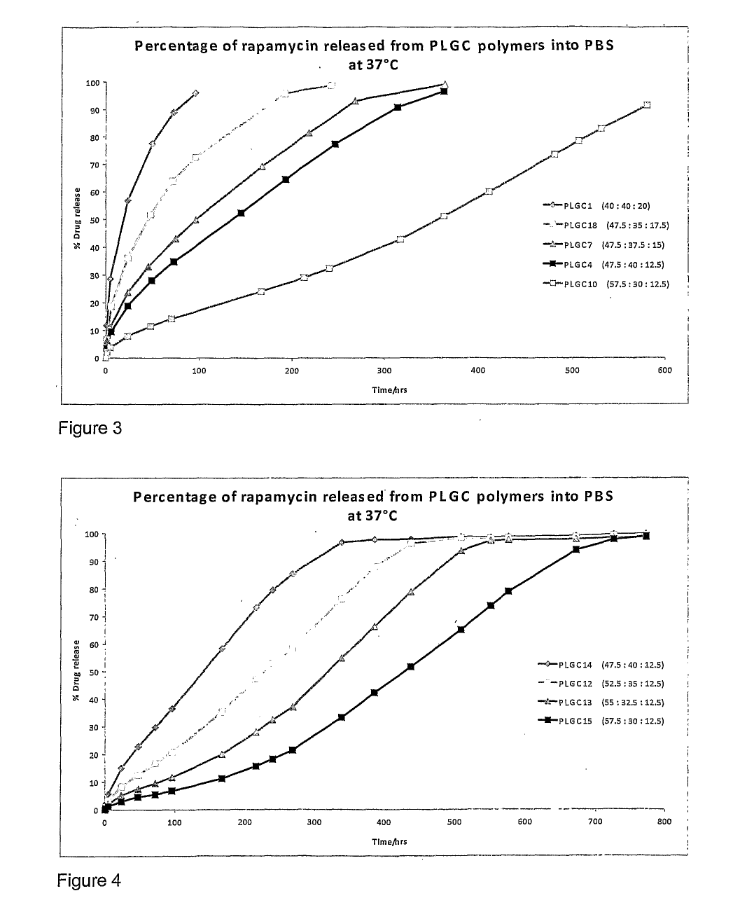

FIG. 3 shows the effect of varying the proportion of ε-caprolactone in the polymer on the release of rapamycin; increasing the fraction of ε-caprolactone increases the rate at which rapamycin is released.

FIG. 4 shows the effect of changing the ratio of D,L-lactide:glycolide with a fixed amount of ε-caprolactone (12.5%) on release of rapamycin; increasing the amount of glycolide increases the rate at which rapamycin is released.

### Example 3

Commercially available stainless steel stents were functionalised, a primer coat applied and overlaid with polymer PLGC1 containing 25% rapamycin in an identical manner to Example 2 Stages 1-5. An “undrugged top coat” of poly(D,L-lactide-co-glycolide) 50:50 (PLGA2) was then applied in the following manner.

The stents were attached to a mandrel and coated on the Sonotek MediCoat Benchtop Coater with a 0.5% w/w solution in CHCl₃ of PLGA2. The parameters used were: 0.09 ml/min flow rate, 1.0 W ultrasonic power, 2 passes, 40 rpm rotation, 0.13 cm/s horizontal travel and 25 mm from stent to spray head. After coating, the stents were dried under vacuum for 16 hours at 40° C. The top-coat made up 10% of the total coating weight.

The elution of rapamycin from the stents was tested in an identical manner to that described in Example 2 Stage 6.

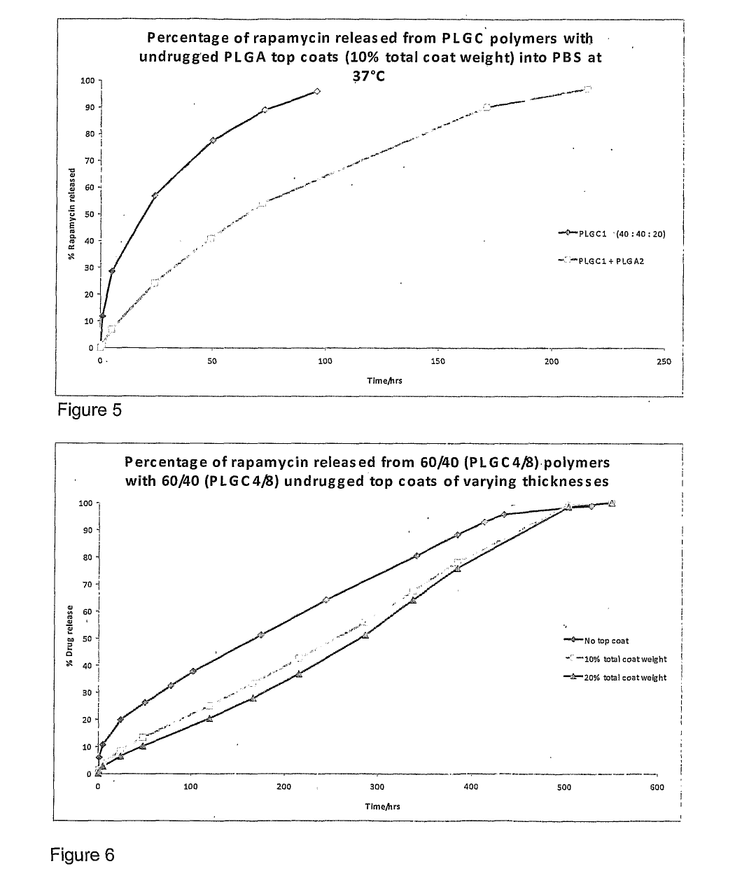

The results, shown in FIG. 5, show that the use of an undrugged top coat can control and slow the release of rapamycin from the coating.

### Example 4

Polymers PLGC4 and PLGC8 were blended together in CHCl₃ in the ratio 60% PLGC4:40% PLGC8 (60/40 PLGC4/8). To this blend was added 25% rapamycin (based on dry weight of polymer i.e. 75% polymer:25% rapamycin).

Commercially available stainless steel stents were functionalised, primed and coated with polymer 60/40 PLGC4/8 containing 25% rapamycin in an identical manner to Example 2 Stages 1-5.

An undrugged top-coat of 60/40 PLGC4/8 was applied in an identical manner to that described in Example 3. The top-coats made up zero, 10 or 20% of the total coating weight.

The elution of rapamycin from the stents was tested in an identical manner to that described in Example 2 Stage 6.

The results, shown in FIG. 6, show how top-coats of different weights can further control drug release, with greater coating weight reducing the rate of elution of rapamycin.

### Example 5

Polymers PLGC4 and PLGC8 were blended together in CHCl₃ with the ratio of PLGC4:PLGC8 varying as follows: 100:0, 80:20, 70:30, 60:40, 50:50. To these blends were added 25% rapamycin (based on dry weight of polymer i.e. 75% polymer:25% rapamycin).

Commercially available stainless steel stents were functionalised, primed and coated with polymer blends containing 25% rapamycin in an identical manner to Example 2 Stages 1-5.

An undrugged top-coat of PLGC4 and PLGC8 blended in the same ratios as their respective main coats was applied in an identical manner to that described in Example 3. The top-coats made up 10% of the total coating weight.

The elution of rapamycin from the stents was tested in an identical manner to that described in Example 2 Stage 6.

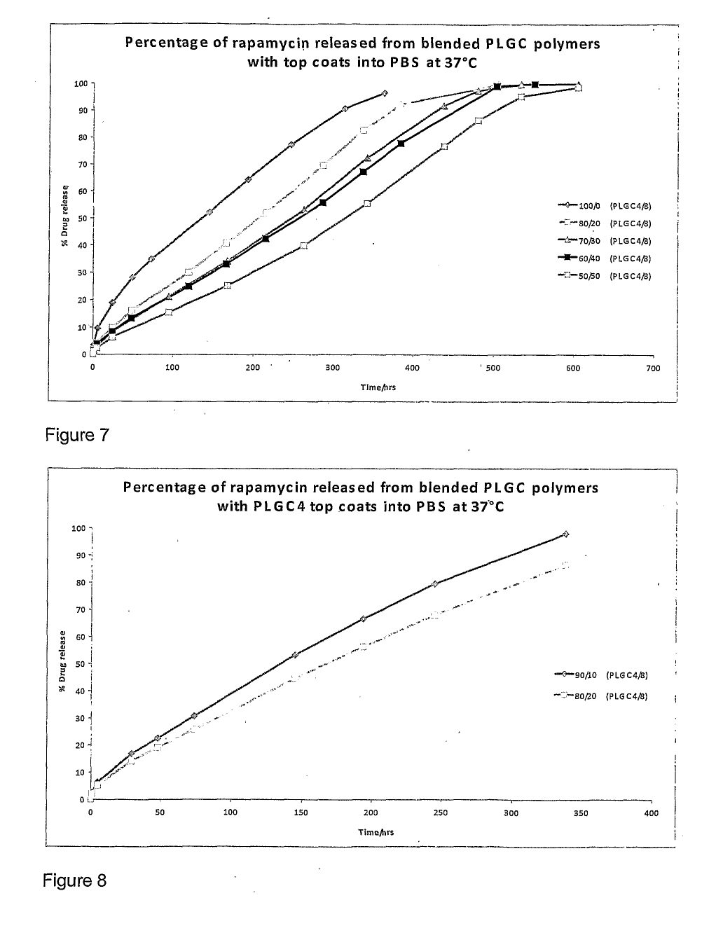

The results, shown in FIG. 7, show how blending of the polymers can be used to control the elution profile of the drug. Specifically, in this case, increasing the ratio of PLGC4 in the blend increases the rate of elution of rapamycin.

### Example 6

Polymers PLGC4 and PLGC8 were blended together in CHCl₃ with the ratio of PLGC4:PLGC8 varying as follows: 90:0, 80:20. To these blends were added 25% rapamycin (based on dry weight of polymer i.e. 75% polymer:25% rapamycin).

Commercially available stainless steel stents were functionalised, primed and coated with polymer blends containing 25% rapamycin in an identical manner to Example 2 Stages 1-5.

An undrugged top-coat of PLGC4 was applied in an identical manner to that described in Example 3. The top-coats made up 10% of the total coating weight.

The elution of rapamycin from the stents was tested in an identical manner to that described in Example 2 Stage 6.

The results, shown in FIG. 8, show again how drug release can be controlled by blending of the polymers.

### Example 7

Polymers PLGC1 and PLGC4 were blended with 25% rapamycin and either 0, 0.75, 1.5 or 3% lauric acid (LA) to produce the following compositions:

| PLGC4 (%) | Rapamycin (%) | Lauric acid (%) |
|---|---|---|
| 75 | 25 | 0 |
| 74.25 | 25 | 0.75 |
| 73.5 | 25 | 1.5 |

| PLGC1 (%) | Rapamycin (%) | Lauric acid (%) |
|---|---|---|
| 75 | 25 | 0 |
| 73.5 | 25 | 1.5 |
| 72 | 25 | 3 |

Commercially available stainless steel stents were functionalised, primed and coated with the above polymer the blends in an identical manner to Example 2 Stages 1-5.

The elution of rapamycin from the stents was tested in an identical manner to that described in Example 2 Stage 6.

The results, shown in FIG. 9, show that lauric acid can be added to the polymer coatings as a degradation accelerant but has a minimal affect on the rate of release of rapamycin.

### Example 8

### Stage 1: Cleaning

Stainless steel samples (50 mm×50 mm×0.25 mm annealed finish) were cleaned by sonication for 15 minutes in a 7.5% w/w solution of aqueous sodium hydrogen carbonate, rinsing in deionised water, sonication for 15 minutes in 2-propanol and sonication for 15 minutes in deionised water. The samples were then dried at 100° C. for 16 hours, followed by drying at 50° C. for 30 minutes.

### Stage 2: Functionalisation

2.5 ml glacial acetic acid solution in toluene (1.0% w/w, 0.4 mmol) was added to 200 ml toluene followed by 1.6 ml TMSPEA and mixed. The samples were removed from the 50° C. oven and immersed in the solution for 5 minutes. The samples were removed and kept at 50° C. for 20 hours.

The samples were rinsed in a series of solvents by rotating sequentially for 15 minutes in each of toluene, methanol, deionised water, methanol and deionised water. Finally, the samples were rinsed for 5 minutes in methanol and then dried at 50° C. for 2 hours.

Anhydrous toluene was added, under nitrogen, into a measuring cylinder being purged with nitrogen. Enough TESPI was added to give a 4% v/v solution in toluene. The samples (dried at 50° C. for 15 minutes and allowed to cool for two minutes before use) were immersed in the solution on a holder and rotated under nitrogen for 15 minutes. The samples were then rinsed in anhydrous toluene under nitrogen and dried under vacuum for 16 hours.

This stage was omitted in half the samples in order to create unfunctionalised control samples for comparison.

### Stage 3: Applying a Primer Coat

The samples were placed at an angle of approximately 45° and primed with 0.5% w/w PLGA1 solutions in CHCl₃. The priming was performed using a handheld spray gun working at 10 psi from a distance of approximately 15 cm. Between 4 and 8 passes were needed, depending on the speed of movement, to achieve a primer coat weight of between 50 and 100 μg per cm2. The samples were then cured for 16 hours at 100° C.

### Stage 4: Coating

After curing at 100° C., the samples were cooled for 5 minutes and then placed at an angle of approximately 45° and coated with a 0.5% w/w PLGC4 solution in CHCl₃. The coating was performed using a handheld spray gun working at 10 psi from a distance of approximately 15 cm. Between 30 and 60 passes were needed, depending on the speed of movement, to achieve a coat weight of 600-700 μg per cm2. The samples were then dried under vacuum at 50° C. for 16 hours.

### Stage 5: Testing

The adhesion tests were performed using the Elcometer 110 PATTI (Pneumatic Adhesion Tensile Test Instrument). Prior to testing all samples were glued to a fixed surface to prevent moving during the procedure. An aluminium pull stub is glued to the test surface using acrylic based super glue. The glue is applied to the stub then placed quickly onto the sample. Taking care to keep the stub still, an activator is sprayed directly onto the interface, whilst simultaneously maintaining pressure on the stub. A pulling piston is attached and a pressurised control module applies increasing pressure to the pull stub until it becomes detached from the test surface. The control module registers the maximum pressure (psig) attained which can be converted into MPa or bond strength (POTS).

The results, shown in FIG. 10, show that functionalising the surface significantly improves the adhesion of the PLGC to the plates'.

### Example 9

### Cleaning

A commercially available stainless steel stent was cleaned by sonication for 15 minutes in a 7.5% w/w solution of aqueous sodium hydrogen carbonate, rinsing in deionised water, sonication for 15 minutes in 2-propanol and sonication for 15 minutes in deionised water. The stent was then dried at 100° C. for 16 hours, followed by drying at 50° C. for 30 minutes.

### Functionalisation

2.5 ml glacial acetic acid solution in toluene (1.0% w/w, 0.4 mmol) was added to 200 ml toluene followed by 1.6 ml TMSPEA and mixed. The stent was removed from the 50° C. oven and immersed in the solution for 5 minutes. The stent was removed and kept at 50° C. for 20 hours.

The stent was rinsed in a series of solvents by rotating sequentially for 15 minutes in each of toluene, methanol, deionised water, methanol and deionised water. Finally, the stent was rinsed for 5 minutes in methanol and then dried at 50° C. for 2 hours.

Anhydrous toluene was added, under nitrogen, into a measuring cylinder being purged with nitrogen. Enough TESPI was added to give a 4% v/v solution in toluene. The stent (dried at 50° C. for 15 minutes and allowed to cool for two minutes before use) was immersed in the solution on a holder and rotated under nitrogen for 15 minutes. The stent was then rinsed in anhydrous toluene under nitrogen and dried under vacuum for 16 hours.

### PLGA Coupling

The stent was attached to a mandrel and coated with a primer solution containing 0.5% w/w PLGA1 in CHCl₃ on a Sonotek MediCoat Benchtop Coater. The parameters used were: 0.075 ml/min flow rate, 0.8 W ultrasonic power, 4 passes, 40 rpm rotation, 0.13 cm/s horizontal travel and 25 mm from stent to spray head. After priming, the stent was left for 16 hours at 100° C.

### Coating

After cooling for 5 minutes, the stent was attached to a mandrel and coated on the Sonotek MediCoat Benchtop Coater with a 0.5% w/w solution in CHCl₃ of PLGC4, PLGC8 and rapamycin (45:30:25). The parameters used were: 0.09 ml/min flow rate, 1.0 W ultrasonic power, 16 passes, 40 rpm rotation, 0.13 cm/s horizontal travel and 25 mm from stent to spray head. After coating, the stent was dried under vacuum for 16 hours at 40° C.

### Top Coat

After cooling for 5 minutes, the stent was attached to a mandrel and coated on the Sonotek MediCoat Benchtop Coater with a 0.5% w/w solution in CHCl₃ of PLGC4 and PLGC8 (60:40). The parameters used were: 0.09 ml/min flow rate, 1.0 W ultrasonic power, 2 passes, 40 rpm rotation, 0.13 cm/s horizontal travel and 25 mm from stent to spray head. After coating, the stent was dried under vacuum for 16 hours at 40° C.

### Tracking and Deployment

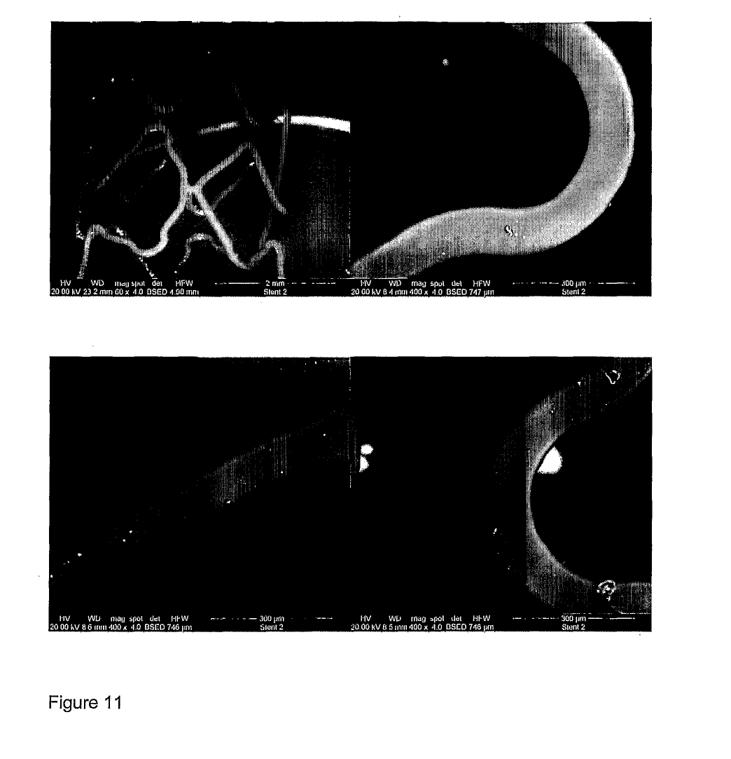

The stent was placed onto the balloon section of a suitably sized commercially available PTCA catheter and crimped by hand. The catheter was fed down a tortuous path in PBS solution at 37° C. to mimic the passage of the stent and catheter in the human arteries. This was repeated five times. Finally, the stent was expanded at the nominal pressure of the device (10 bar). The expanded stent was rinsed in deionised water, dried and observed under an optical microscope. No pitting, cracking or delamination of the coating was observed (see FIG. 11), including at the expansion points of the stent struts.

### Example 10

Polymers were prepared following the same methods as described in Example 1 using the ratios of lactide, glycolide and ε-caprolactone shown below:

| | Composition (%) | | |
|---|---|---|---|
| Ref Code | Lactide | Glycolide | Caprolactone |
| PLGC21 | 77.5 | 10 | 12.5 |
| PLGC22 | 75 | 10 | 15 |
| PLGC23 | 76.75 | 10 | 13.25 |

Stents were cleaned, functionalised and a primer coat of PLGA1 applied as described in Example 2 Stages 1-3. The stents were then coated with polymers PLGC21, PLGC22 and PLGC23 containing 25% rapamycin as described in Example 2, Stages 4 and 5. The elution of rapamycin from the stents was tested in an identical manner to that described in Example 2, Stage 6.

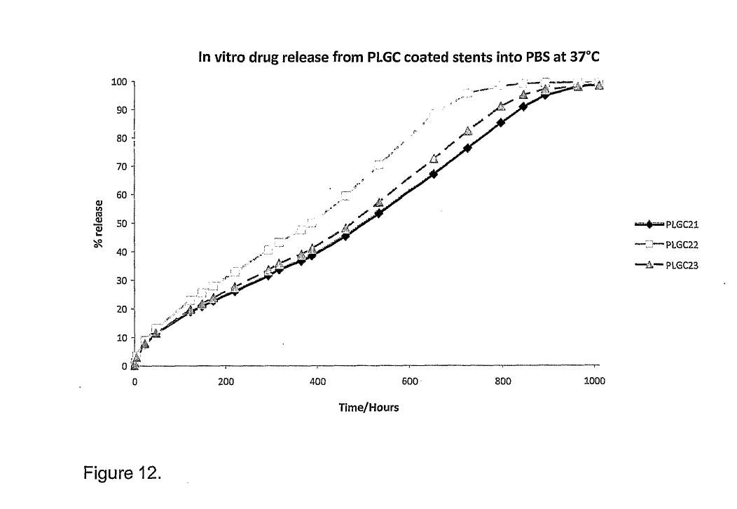

The results, shown in FIG. 12, show that the upturn in release caused by the onset of degradation can be delayed, and drug can be released over a longer timescale, with a lower glycolide content. Furthermore for a constant glycolide content, drug release can be extended by lowering the caprolactone content. In this example drug release can be extended to around 1000 hours (42 days).

Overall, the system described can be designed to tailor drug release profiles between about 100 and 1000 hours with a reasonably consistent rate of drug release.

What is claimed is:

1. A bioresorbable coating composition for an implantable medical device, wherein the composition comprises a co-polymer of a lactide, a glycolide and ε-caprolactone and at least one drug and/or bioactive agent.

2. The bioresorbable coating composition of claim 1, wherein the composition comprises a glycolide at between about 30% to about 45% by weight of the polymer component of the composition.

3. The bioresorbable coating composition of claim 1, wherein the composition comprises a glycolide at between about 5% to about 15% by weight of the polymer component of the composition.

4. The bioresorbable coating composition of claim 3, wherein the composition comprises a glycolide at about 10% by weight of the polymer component of the composition.

5. The bioresorbable coating composition of claim 3, wherein the composition comprises ε-caprolactone at between about 10% and about 20% by weight of the polymer component of the composition.

6. The bioresorbable coating composition of claim 5, wherein the composition comprises ε-caprolactone at about 12.5% by weight of the polymer component of the composition.

7. (canceled)

8. The bioresorbable coating composition of claim 1, wherein the lactide is D,L-lactide.

9. The bioresorbable coating composition of claim 1, wherein the composition comprises a blend of co-polymers.

10. The bioresorbable coating composition according to claim 9, wherein a second copolymer of the blend is a co-polymer of a lactide, a glycolide and ε-caprolactone.

11. The bioresorbable coating composition of claim 10, wherein the blend comprises a first co-polymer comprising 47.5% D,L-lactide, 40% glycolide and 12.5% ε-caprolactone and a second co-polymer comprising 57.5% D,L-lactide, 30% glycolide and 12.5% ε-caprolactone.

12. The bioresorbable coating composition of claim 1, wherein the at least one drug and/or bioactive agent is an immunosuppressant.

13. The bioresorbable coating composition of claim 12, wherein the immunosuppressant is rapamycin.

14. The bioresorbable, coating composition of claim 1, wherein the at least one drug and/or bioactive agent is an antibiotic.

15. The bioresorbable coating composition of claim 14, wherein the antibiotic is vancomycin.

16. An implantable medical device coated with a bioresorbable coating composition wherein the composition comprises a co-polymer of a lactide, a glycolide and ε-caprolactone and at least one drug and/or bioactive agent.

17. The implantable medical device of claim 16, wherein the bioresorbable coating composition is chemically and/or physically coupled to at least part of a surface of the implantable medical device.

18. The implantable medical device of claim 17, wherein a primer coat is provided adjacent to a surface of the device and the bioresorbable coating polymeric composition overlays the primer coat.

19. The implantable medical device of claim 18, wherein the molecular weight of the primer coat is lower than the molecular weight of the bioresorbable coating polymeric composition.

20. The implantable medical device claim 18, wherein the primer coat comprises a co-polymer of a lactide and a glycolide.

21. The implantable medical device of claim 20, wherein the co-polymer is poly(D,L-lactide-co-glycolide).

22. The implantable medical device of claim 21, wherein the ratio of D₁L-lactide to glycolide is 50:50.

23. The implantable medical device of claim 22, wherein the molecular weight of the copolymer is between about 5-15 k.

24. The implantable medical device of claim 23, wherein the bioresorbable coating composition comprises a glycolide at about 10% by weight of the polymer component of the composition and ε-caprolactone at about 12.5% by weight of the polymer component of the composition and wherein the primer coat comprises poly(D,L-lactide-co-glycolide at a 50:50 ratio of D,L-lactide: glycolide and having a molecular weight of between about 5-15 k.

25. The implantable medical device of claim 18, wherein a top coat is applied to the bioresorbable coating composition.

26. The implantable medical device of claim 25, wherein the top coat does not comprise at least one drug and/or bioactive agent

27. The implantable medical device of claim 25, wherein the top coat comprises a co-polymer of a lactide and a glycolide.

28. The implantable medical device of claim 27, wherein the copolymer is poly(D,L-lactide-co-glycolide).

29. The implantable medical device of claim 28, wherein the ratio of D₁ L-lactide to glycolide is 50:50.

30. (canceled)

31. (canceled)

32. The implantable medical device of claim 25, wherein the top coat is less than 50% of the total coating.

33. The mplantable medical device of claim 32, wherein the top coat is between about 10%-20% of the total coating.

34. (canceled)

35. (canceled)

36. A method of applying a bioresorbable coating composition to an implantable medical device comprising the steps of;
i. providing an implantable medical device,
ii. applying a coating of a bioresorbable coating composition to at least part of the surface of the device wherein the composition comprises a co-polymer of a lactide, a glycolide and εcaprolactone and at least one drug and/or bioactive agent.

37. The method according to claim 36, wherein the method further comprises applying a primer coat to at least a part of the surface of the device and overlaying this coating with a coating of the bioresorbable coating composition.

38. The method of claim 37, wherein the method further comprises applying a top coat wherein the top coat comprises a co-polymer of a lactide and a glycolide.

39. (canceled)

40. (canceled)

41. (canceled)
